(19) United States
(12) Patent Application Publication
(10) Pub. No.: US 2026/0341535 A1
(43) Pub. Date: Oct. 29, 2026

(54) INFORMATION PROVIDING DEVICE, INFORMATION PROVIDING METHOD, AND RECORDING MEDIUM

(71) Applicant: NEC corporation, Tokyo (JP)

(72) Inventors: Yutaka UNO, Tokyo (JP); Masanori Tsujikawa, Tokyo (JP); Soma Onishi, Tokyo (JP); Daisaku Shibaa, Tokyo (JP); Junya Inoh, Tokyo (JP)

(73) Assignee: NEC Corporatioln, Tokyo (JP)

(21) Appl. No.: 19/571,845

(22) Filed: Mar. 19, 2026

(30) Foreign Application Priority Data
Mar. 26, 2025 (JP) 2025-051720

(51) Int. Cl. G16H 70/20

(52) U.S. Cl. CPC G16H 70/20

(57) ABSTRACT

In the information providing device, the operation log generation unit generates an operation log of the user in a case where the user accesses the medical information. The conversation log generation unit generates a conversation log between the user and an AI agent for supporting provision of the medical information. The condition acquisition unit acquires the conditions at the start of the operation by the user. The prediction unit predicts information to be provided to the user from the identification information of the user and the conditions at the start of the operation by the user using a prediction model trained in advance using the operation log and the conversation log. The proposal unit proposes, to the user, provision of the information to be provided.

### INCORPORATION BY REFERENCE

This application is based upon and claims the benefit of priority from Japanese Patent Application 2025-051720, filed on Mar. 26, 2025, the disclosure of which is incorporated herein in its entirety by reference.

### TECHNICAL FIELD

The present disclosure relates to a technology for providing information required by a user.

### BACKGROUND ART

There is known a medical information system that includes a relational database (RDB) storing medical information and provides information related to various hospital tasks. JP2015-170214A describes an example of a database system that provides medical information.

### SUMMARY

The tasks of medical personnel are often performed in a fixed routine to some extent. In such cases, a user such as a doctor or a nurse who uses a medical information database (hereinafter, a database is referred to as a “DB”) must repeat similar operations when acquiring information necessary for the task to be performed or registering new information acquired through an examination or the like, which is cumbersome.

One object of the present disclosure is to provide an information providing device capable of predicting and providing information required by a user according to a condition when the user accesses a DB.

According to an example aspect of the present invention, there is provided an information providing device comprising:
an operation log generation means for generating an operation log of a user in a case where the user accesses medical information;
a conversation log generation means for generating a conversation log between the user and an AI agent that supports provision of medical information;
a condition acquisition means for acquiring a condition at a start of an operation by the user;
a prediction means for predicting information to be provided to the user from identification information of the user and the condition at the start of the operation by the user using a prediction model trained in advance using the operation log and the conversation log; and
a proposal means for proposing, to the user, provision of the information to be provided.

According to another example aspect of the present invention, there is provided an information providing method executed by a computer, the information providing method comprising:
generating an operation log of a user in a case where the user accesses medical information;
generating a conversation log between the user and an AI agent that supports provision of medical information;
acquiring a condition at the start of an operation by the user;
predicting information to be provided to the user from identification information of the user and the condition at the start of the operation by the user using a prediction model trained in advance using the operation log and the conversation log; and
proposing, to the user, provision of the information to be provided.

According to still another example aspect of the present invention, there is provided a program that causes a computer to execute processing comprising:
generating an operation log of a user in a case where the user accesses medical information;
generating a conversation log between the user and an AI agent that supports provision of medical information;
acquiring a condition at the start of an operation by the user;
predicting information to be provided to the user from identification information of the user and the condition at the start of the operation by the user using a prediction model trained in advance using the operation log and the conversation log; and
proposing, to the user, provision of the information to be provided.

According to the present disclosure, it is possible to predict and provide information required by a user according to a condition when the user accesses a DB.

### BRIEF DESCRIPTION OF THE DRAWINGS

FIG. 1 illustrates an entire configuration of an information providing device according to the present disclosure;

FIG. 2 is a block diagram illustrating a hardware configuration of the information providing device;

FIG. 3 is a block diagram illustrating a functional configuration of the information providing device;

FIG. 4 illustrates a display example of an operation screen in a case where a user accesses medical information;

FIG. 5 illustrates another display example of an operation screen in a case where a user accesses medical information;

FIG. 6 is a flowchart of information collection processing by the information providing device;

FIG. 7 is a flowchart of information providing processing by the information providing device;

FIG. 8 is a block diagram illustrating a functional configuration of another information providing device according to the present disclosure; and

FIG. 9 is a flowchart of processing that the information providing device performs.

### EXAMPLE EMBODIMENTS

Preferred example embodiments of the present disclosure will be described below with reference to the drawings.
The tasks of medical personnel are often performed in a fixed routine to some extent. For example, doctors often have fixed days and time slots for examining patients on a weekly schedule, and nurses also often perform patient rounds and record the results at fixed times each day. In such cases, a user such as a doctor or a nurse who uses a medical information DB must repeat similar operations when acquiring information necessary for a task to be performed or registering new information acquired through an examination or the like, which is cumbersome.

In addition, the user of the medical information DB often has characteristics such as individual preferences and habits in selection of a graphical user interface (GUI), an operation procedure, and the like when viewing target information. Therefore, it is possible to predict what kind of operation the user performs and what kind of information the user is seeking to some extent based on these characteristics.

Therefore, the present example embodiments prepare a prediction model for predicting information desired by the user based on a past operation record by the user on the medical information DB and a condition under which the user accessed the medical information DB, and proposes provision of the predicted information to the user. As a result, for example, in a case where a certain doctor accesses the medical information DB in a certain time slot on a certain day of the week, the prediction model can predict that the doctor will check the medical record in advance for examination of a patient scheduled thereafter, and propose display of the electronic medical record of the patient to be examined.

An example of the past operation record includes an operation log of a GUI for the user to operate an electronic medical record or the like registered in the medical information DB. In this case, an information extraction log from the medical information DB generated as a result of an operation indicated by an operation log may be included in the operation log and used. In addition, in a case where an artificial intelligence (AI) agent that conversationally supports the user’s access to medical information is prepared, the conversation log can be used as an operation record. In this case, an information extraction log from the medical information DB generated as a result of a conversation indicated by a conversation log may be included in the conversation log and used.

These operation logs and conversation logs often reflect the characteristics such as preferences and habits of a user, such as a doctor, in performing a task or in an operation procedure for acquiring information. Therefore, by proposing the provision of information expected to be required by an individual user based on the user and the conditions under which the user is accessing the medical information DB, the user can carry out the medical task more efficiently.

### First Example Embodiment

### Overall Configuration

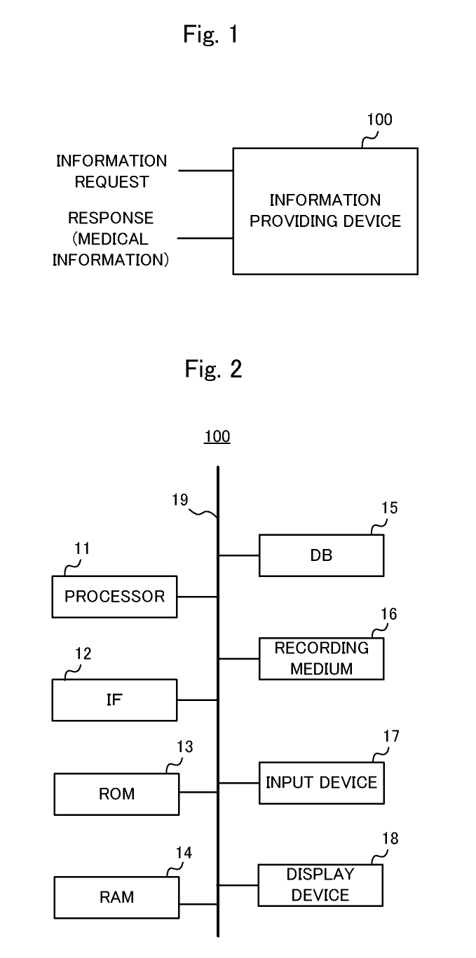

FIG. 1 illustrates an entire configuration of an information providing device according to the present disclosure. An information providing device 100 is provided in a hospital or the like, and provides medical information to a user such as a doctor or a nurse working in the hospital. The user inputs an information request to the information providing device 100. For example, the information request is for information regarding a specific patient, or related information useful for examining or treating the patient.

Generally, the user can access the target information by operating a GUI for browsing information. Further, in the information providing device 100, a conversational AI agent for supporting the user’s access to medical information is prepared, and the user can make an information request using natural language. That is, instead of inputting an information request in a predetermined format according to a specific GUI or the like, the user can express what kind of information the user wants in words to input the information. The conversational AI agent interprets the natural language information request input by the user, and displays the requested medical information as a response on the screen of the display device. As a result, it is possible to provide the required information in response to the natural language information request.

In the present example embodiment, the information providing device 100 uses a relational database (hereinafter also referred to as “RDB”) that stores medical information. The RDB is a database that manages data in a table format. In the RDB, data is stored in a plurality of tables, and a large number of pieces of data are stored by associating the tables. Note that a system that stores, operates, controls, or otherwise processes information using the relational database is referred to as a relational database management system (RDBMS). The information providing device 100 is an example of an RDBMS.

### Hardware Configuration

FIG. 2 is a block diagram illustrating a hardware configuration of the information providing device 100. As illustrated, the information providing device 100 includes a processor 11, an interface (IF) 12, a read only memory (ROM) 13, a random access memory (RAM) 14, a database (DB) 15, a storage medium 16, an input device 17, and a display device 18. The components are connected to each other via a bus 19, for example.

The processor 11 is a computer such as a Central Processing Unit (CPU), and controls an entirety of the information providing device 100 by executing a program prepared in advance. Specifically, as the processor 11, a CPU, a graphics processing unit (GPU), a digital signal processor (DSP), a micro processing unit (MPU), a floating point unit (FPU), a physics processing unit (PPU), a tensor processing unit (TPU), a quantum processor, a microcontroller, or a combination of these can be used.

In addition, the processor 11 loads a program stored in the ROM 13 or the storage medium 16 into the RAM 14 and executes each process coded in the program. The processor 11 functions as a part or all of the information providing device 100. Specifically, the processor 11 performs information collection processing and information providing processing, which will be described later.

The IF 12 transmits and receives data to and from an external device. Specifically, the information providing device 100 receives an information request from the user through the IF 12. Further, the information providing device 100 can output the requested information to a storage device or an external device through the IF 12 as necessary.

The ROM 13 stores various programs to be executed by the processor 11. The RAM 14 is used as a working memory during the execution of various pieces of the processing by the processor 11.

The DB 15 includes an RDB that stores medical information as described above. In addition, the DB 15 stores various algorithms, data, a machine learning model, a deep learning model, and the like used when the information providing device 100 executes the information collection processing and information providing processing to be described later.

The storage medium 16 is a non-volatile non-transitory storage medium such as a disk-shaped recording medium or a semiconductor memory. The storage medium 16 may be detachably attached to the information providing device 100. The storage medium 16 records various programs to be executed by the processor 11.

The input device 17 is a keyboard, a mouse, a touch panel, or the like, and is mainly used when a user inputs an information request. The display device 18 is a liquid crystal display or the like, and is mainly used to display information requested by the user.

### Functional Configuration

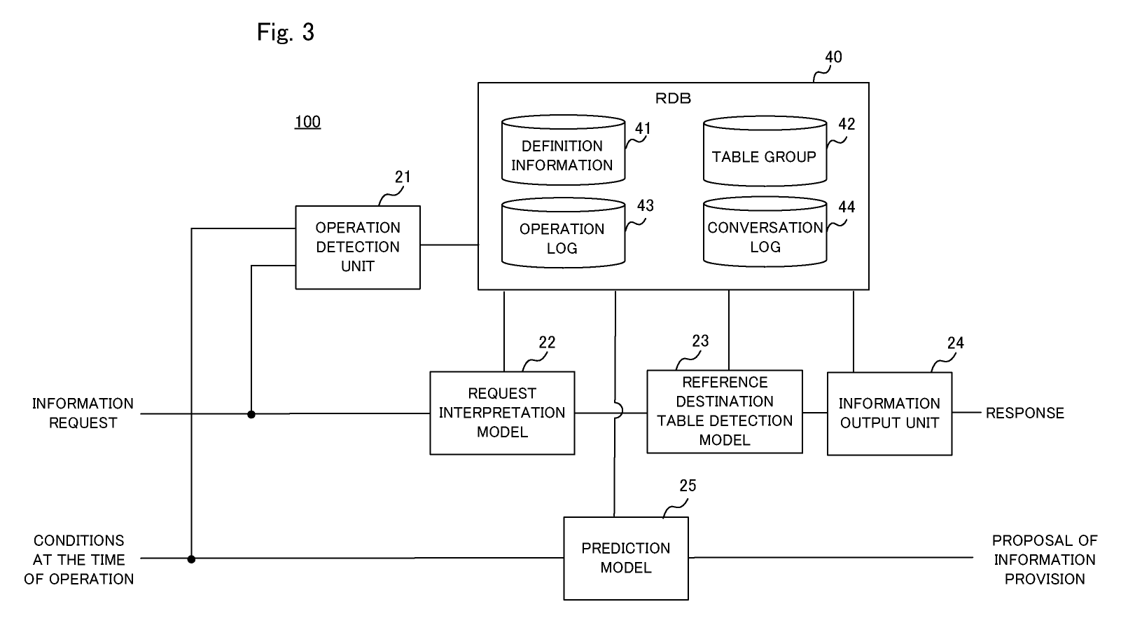

FIG. 3 is a block diagram illustrating a functional configuration of the information providing device 100. The information providing device 100 functionally includes an operation detection unit 21, a request interpretation model 22, a reference destination table detection model 23, an information output unit 24, a prediction model 25, and an RDB 40. The operation detection unit 21, the request interpretation model 22, the reference destination table detection model 23, the information output unit 24, and the prediction model 25 are implemented by the processor 11 illustrated in FIG. 2. The RDB 40 is implemented by the DB 15 illustrated in FIG. 2.

First, the RDB 40 will be described. The RDB 40 includes definition information 41, a table group 42, an operation log 43, and a conversation log 44. An RDB is a type of DB that stores data in a plurality of table formats, and medical information is stored in the table group 42. The definition information 41 includes a master table and a table definition. The master table is a table that manages master data which is basic data used in the RDB 40, and manages data shared throughout the RDB 40 such as “doctor information,” “patient information,” and “disease information.” The table definition is information that defines a structure of a table that stores and manages data in the RDB 40, and includes, for example, definitions related to the table name, columns, a primary key, a foreign key, and the like of each table. The table group 42 includes a plurality of tables in which medical information to be stored is stored in a table format.

The operation log 43 is an operation history of a GUI used by a user to access the medical information in the RDB 40. The conversation log 44 is a conversation record with a conversational AI agent that supports the user conversationally. Note that the conversational AI agent includes the request interpretation model 22, the reference destination table detection model 23, and the information output unit 24.

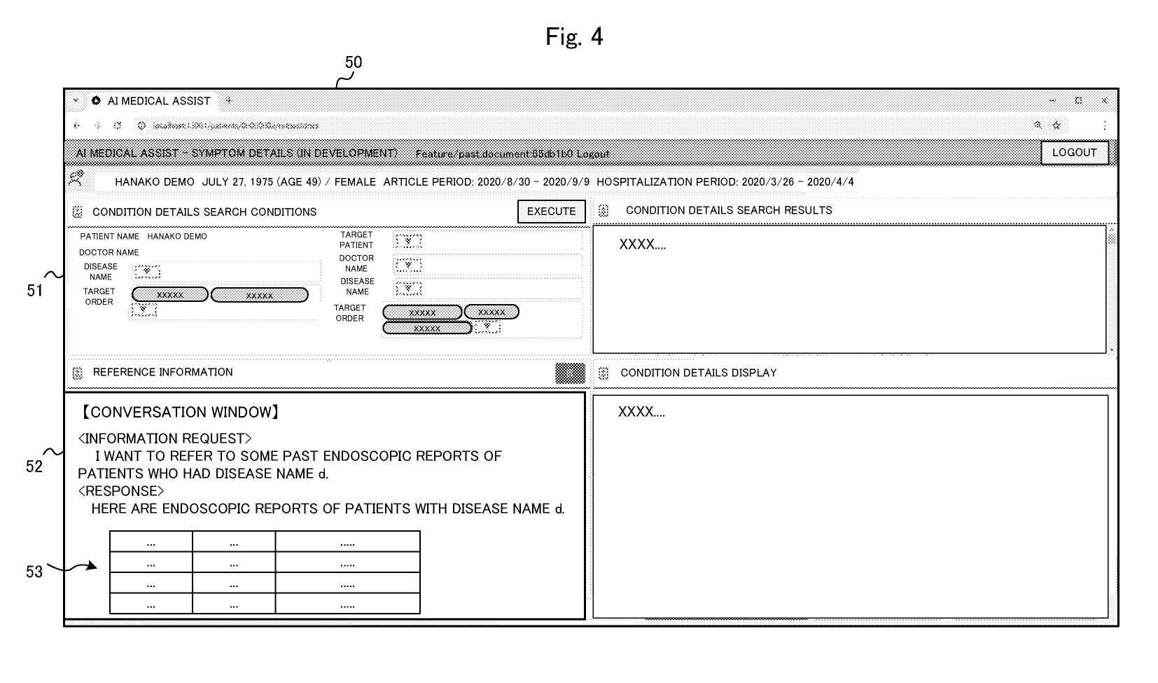

The operation detection unit 21 receives an information request by the user, and detects an operation performed on the GUI by the user. FIG. 4 illustrates a display example of a GUI when the user accesses the medical information. Specifically, FIG. 4 is an example of an operation screen displayed on a display device 18. An operation screen 50 includes a search window 51 and a conversation window 52. The user can access the desired medical information by inputting a search condition in the search window 51. Furthermore, the user can access the desired medical information by inputting a sentence or the like specifying the desired information in natural language to the conversation window 52. In the example of FIG. 4, the user has input the information request “I want to refer to some endoscopy reports of past patients who had disease name d and symptom s.” In response, the information providing device 100 displays requested information 53 together with a sentence “Please refer to the following table.”

The operation detection unit 21 detects the content input by the user to the search window 51, the order in which the content was input, and the like as the user’s operation. In addition, the operation detection unit 21 detects the content input by the user to the conversation window 52, the order in which the content was input, and the like as the user’s operation. Note that, in the information providing device 100, in a case where a plurality of operation screens (GUIs) are prepared so as to be selectable in the initial menu, and the user selects one of the operation screens and reaches the operation screen in FIG. 4, the operation detection unit 21 detects information of the operation screen (GUI) selected by the user, the order of the series of operations until the operation screen was reached, and the like as the user’s operation.

In addition, conditions at the time of operation are input to the operation detection unit 21. The conditions at the time of operation represent information indicating the conditions and situation when the user operated the information providing device 100. Specifically, the conditions at the time of operation include identification information of the user (doctor or nurse), operation date and time (year, month, day, time), an identification number of the terminal device used for the operation, and the like.

The operation detection unit 21 records the detected user’s operation in the operation log 43 of the RDB 40 in association with the conditions at the time of operation. As a result, in the operation log 43, the patient’s disease, medical department, medical task performed by the user, information provided to the user, and the like are recorded in association with the identification information of the user and the operation date and time. In addition, the operation detection unit 21 can record a series of operations performed by the user at the operation date and time as one operation sequence by recording the order in which operations were performed by the user. Each time a user accesses the medical information, the operation detection unit 21 detects the operations by the user and the conditions at the time of the operations, and records them in the operation log 43.

The request interpretation model 20 interprets the information request input by the user and identifies the information requested by the user. Specifically, in a case where the user inputs a search character string in the search window of the operation screen, the request interpretation model 22 uses information indicated by the search character string as request information. On the other hand, in a case where the user inputs an information request in natural language in the conversation window of the operation screen, the request interpretation model 22 interprets the input in natural language to identify the information required by the user. The request interpretation model 22 uses a language model such as a large language model (LLM). For example, it is assumed that the user makes the natural language information request “I want to refer to some endoscopic reports of past patients who had disease name d and symptom s.” In this case, the request interpretation model 22 interprets the content of the information request by a language model such as an LLM, and generates request information as follows.

### <Request Information>

• Information desired to be referred to: endoscope report
• Conditions for referencing: disease name d, symptom s, endoscopy
The request interpretation model 22 records the acquired request information in the operation log 43 and outputs the request information to the reference destination table detection model 23.

The reference destination table detection model 23 detects where in the RDB 40 the input request information exists, that is, the location of the request information. Specifically, the reference destination table detection model 23 refers to the definition information 41 of the RDB 40 and the table group 42 to determine which row of which table the request information exists within the RDB 40. Then, the reference destination table detection model 23 records the location of the detected request information in the operation log 43 and outputs the location to the information output unit 24.

Note that the reference destination table detection model 23 may extract medical entities of the request information input from the request interpretation model 22 and detect the location of the request information also using the medical entities. The medical entity is information representing an idea, an organization, a person, a target, or the like indicated by each piece of information included in the request information. For example, human-related medical entities include patients, doctors, nurses, and the like, medical data-related medical entities include diagnoses, prescriptions, tests, symptoms, and the like, and disease/treatment-related medical entities include diseases, treatments, surgeries, and the like. Generally, in medical information systems, medical information is classified by medical entity and stored. Therefore, the reference destination table detection model 23 can identify the location of the request information more easily by searching the RDB 40 using the medical entities.

The reference destination table detection model 23 searches the RDB 40 for the target information, and it is preferable to use embedding vectors for this search. This is because the name (e.g., a disease name or the like) input as the information request by the user does not necessarily coincide with the information registered in the RDB 40. That is, while disease names and the like are registered in the RDB 40 in professional expressions, official names, and the like, the disease names input by a doctor or a nurse who is the user are often relatively general expressions, and often do not match by a simple character string comparison. Therefore, the reference destination table detection model 23 preferably calculates the similarity (e.g., cosine similarity or the like) between the embedding vector of a word included in the request information and the embedding vector of information registered in the RDB 40, and detects the reference destination table in the RDB 40 in which the information included in the request information exists.

The reference destination table detection model 23 may determine the reference destination after narrowing down the search targets by means of a vector nearest-neighbor search index using an embedding vector of a request word and an embedding vector of, for example, a table definition/column name/representative value. As a result, the frequency of full-text search for a fuzzy input can be reduced, and a search delay can be suppressed.

The information output unit 24 acquires the request information from the reference destination table detected by the reference destination table detection model 23, and outputs the request information as a response to the information request by the user. Specifically, the information output unit 24 creates an SQL query for acquiring a record indicated by the reference destination table from the RDB 40. An SQL query is a command described according to Structured Query Language (SQL) which is a language for operating RDBs, and is used to acquire, add, update, delete, and otherwise process data in RDBs. Then, the information output unit 24 executes the created SQL query, acquires the request information from the RDB 40, and outputs the request information as a response to the information request by the user.

As described above, in a case where the user inputs search conditions to the search window 51 of the operation screen, the operation detection unit 21 records the operation of the user and the conditions at the time of the operation in the operation log 43, and the information output unit 24 records the response output to the user in the operation log 43. In addition, in a case where the user inputs an information request in natural language into the conversation window 52 of the operation screen, the operation detection unit 21 records the operation of the user and the conditions at the time of the operation in the operation log 43. The request interpretation model 22 records the interpreted request information of the user in the operation log 43, and the information output unit 24 records the response output to the user in the operation log 43. In addition, the operation detection unit 21, the reference destination table detection model 23, and the information output unit 24 operate as a conversational AI agent, and record the conversation with the user (i.e., the input by the user and the response output to the user) in the conversation log 44. In this way, each time a user operates the operation screen to access the target information, the operations and the conversation at that time are accumulated in the RDB 40.

The prediction model 25 is a model trained using the operation log 43 and/or the conversation log 44 described above. When the user opens the operation screen and starts an operation related to a medical task, the information providing device 100 acquires the conditions at the time of operation and inputs the conditions to the prediction model 25. The prediction model 25 predicts the information required by the user at that time based on the input conditions, and proposes provision of the information.

The prediction model 25 can be a machine learning model pre-trained using past operation logs and/or conversation logs. In one example, a rule-based prediction model can be generated based on past operation logs and/or conversation logs. In that case, a rule is created that defines information requested by a user, sequences of tasks performed by the user, and the like in association with combinations of identification information of the user and conditions at the time of operation (such as the date and time of operation), and the prediction model predicts the information to be provided by referring to the rule.

In another example, a deep learning model may be used to generate a prediction model. In that case, using the identification information of the user and the conditions at the time of operation as inputs, training data is prepared based on past operation logs and/or conversation logs, where the information output to the user at that time is the ground truth. Then, the deep learning model may be trained by supervised learning using this training data to generate a trained prediction model.

In still another example, a plurality of prediction models may be prepared in association with conditions such as identification numbers of users, and dates and times, and when a user starts operating the information providing device 100, a corresponding prediction model may be selected from the prediction models based on the date and time at that time and used. As described above, in the present example embodiment, the configuration and type of the prediction model are not particularly limited.

Based on the reference destination identified by the reference destination table detection model 23, the information output unit 24 generates a query including a joining condition in a case where a plurality of table references are required, and issues an integrated query in response to a plurality of requests issued within the same session, thereby reducing the number of round trips to the database.

Further, at the time the prediction model 25 predicts a candidate for the information to be provided, the information providing device 100 may acquire a record or summary information corresponding to the candidate in the background and store it in a per-session cache area. Accordingly, in a case where the user agrees with the proposal, the display can be completed without additionally issuing a database access or only with limited difference acquisition.

Note that the information acquired in the background is not output to the display device until the user’s consent is obtained, and is held in the internal cache. Here, a session refers to a period containing a series of operations by the user from activation to termination of the operation screen.

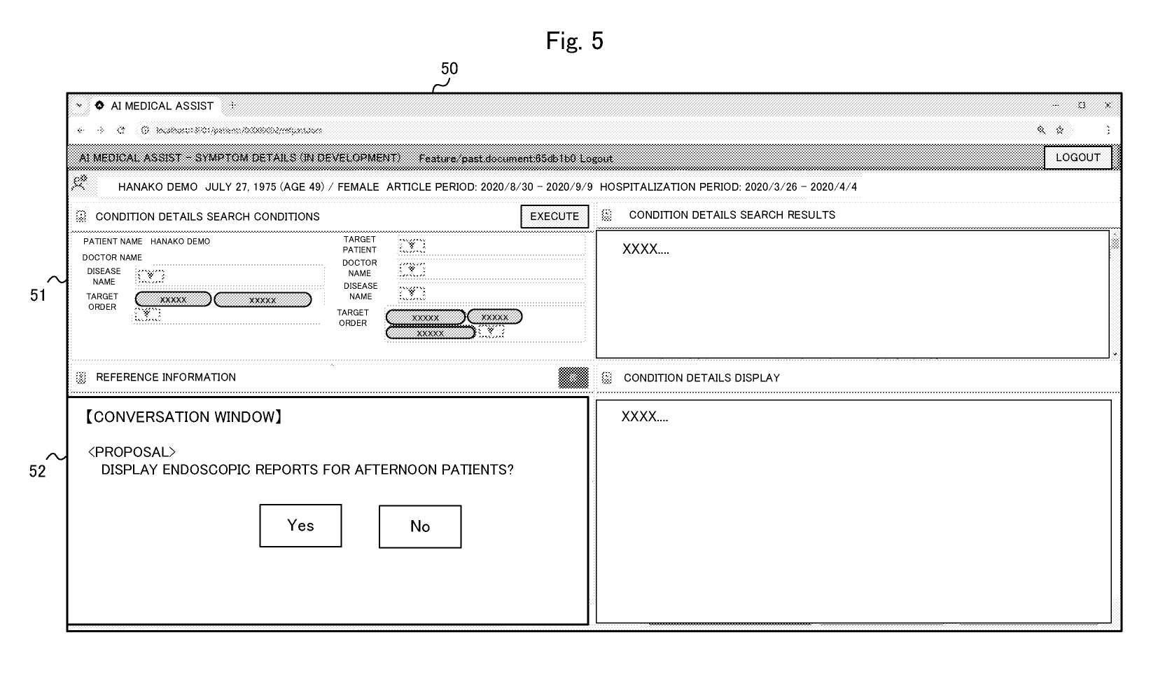

FIG. 5 illustrates a display example in a case where information predicted by the prediction model 25 is proposed. For example, it is assumed that a gastroenterologist examines patients every Monday afternoon, and thus checks endoscope reports of the patients every Monday morning. In this case, when the doctor operates the information providing device 100 to display the operation screen in the morning on Monday, the information providing device 100 acquires the identification information of the doctor, the date and time at that time, and the like as the conditions at the time of operation, and inputs the acquired information to the prediction model 25. Based on the input information, the prediction model 25 predicts that the doctor would like to view the patients’ endoscopic reports and proposes information provision. For example, as illustrated in FIG. 5, the prediction model 25 displays a proposal “Display endoscopic reports for the afternoon patients?” and Yes and No buttons.

In a case where the doctor presses the Yes button and agrees, the information providing device 100 acquires the proposed “endoscopic reports for afternoon patients” from the RDB 40 and displays the acquired reports on the conversation window 52. On the other hand, when the doctor presses the No button, the information providing device 100 may display “What kind of information are you looking for?” or the like to prompt the doctor to make an information request to the conversation window 52. Furthermore, in a case where the prediction model 25 predicts a plurality of pieces of information as the information to be provided based on the conditions at the time of operation by the doctor, the information providing device 100 may propose the pieces of information one by one in descending order of prediction score, or may display a list of the pieces of information to allow the user to select the desired information. As described above, when the user operates the information providing device 100 to perform a routine medical task performed monthly or on a specific weekday, the information providing device 100 predicts the information required by the user based on the conditions at that time and proposes provision of the information. As a result, the user can acquire desired information without having to input the same information request every time.

In the above configuration, the operation detection unit 21 is an example of an operation log generation means and a condition acquisition means; the request interpretation model 22; the reference destination table detection model 23, and the information output unit 24 are examples of a conversation log generation means; and the prediction model 25 is an example of a prediction means and a proposal means.

### Information Collection Processing

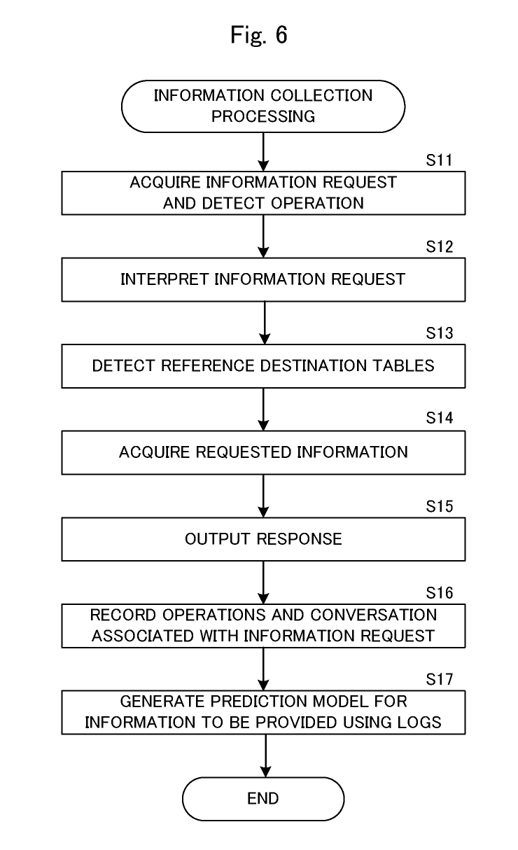

FIG. 6 is a flowchart of information collection processing by the information providing device 100. The information collection processing is processing in which the information providing device 100 records an operation log and/or a conversation log based on a user’s information request and generates the prediction model 25. This processing is implemented by the processor 11 illustrated in FIG. 2 executing a program prepared in advance and operating as each element illustrated in FIG. 3.

First, when the user inputs an information request, the operation detection unit 21 detects an operation associated therewith (step S11). Next, the request interpretation model 20 interprets the information request and generates the request information (step S12). Next, the reference destination table detection model 23 refers to the RDB 40 and detects a reference destination table in which the information included in the request information exists (step S13). Next, the information output unit 24 creates and executes an SQL query to acquire the request information from the reference destination table (step S14), and outputs the request information as a response to the user (step S15).

Next, the operation detection unit 21, the request interpretation model 22, the information output unit 24, and the like record a series of operations from the information request by the user to the response, and their order in the operation log 43, and record the conversation by the conversational AI agent in the conversation log 44 (step S16). Then, a prediction model is generated using the recorded operation log 43 and/or conversation log 44 (step S17).

### Information Providing processing

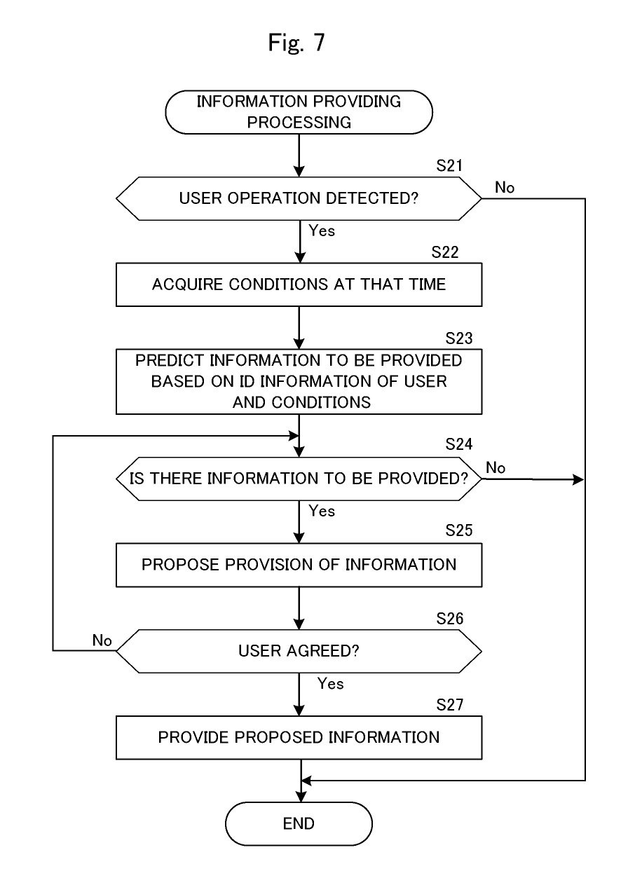

FIG. 7 is a flowchart of information providing processing by the information providing device 100. The information providing processing is processing in which the information providing device 100 predicts the information requested by the user when the user starts operating the information providing device 100, and proposes provision of the information. This processing is implemented by the processor 11 illustrated in FIG. 2 executing a program prepared in advance and operating as each element illustrated in FIG. 3.

When the user starts operating the information providing device 100, the operation detection unit 21 determines whether an operation by the user has been detected (step S21). When no operation by the user is detected (step S21: No), the processing ends. On the other hand, in a case where an operation by the user is detected (step S21; Yes), the information providing device 100 acquires conditions at that time, that is, the identification information of the user, date and time, and the like (step S22). Next, the prediction model 25 predicts the information to be provided based on the identification information of the user and the conditions (step S23). In a case where there is information to be provided (step S24: Yes), the information providing device 100 proposes provision of the information as in the display example of FIG. 5 (step S25), and determines whether the user has agreed (step S26). When the user agrees (step S26: Yes), the information providing device 100 provides the proposed information (step S27), and the information providing processing ends.

On the other hand, in a case where the user does not agree (step S25: No), the information providing device 100 determines whether there is other information to be provided (step S24). For example, in a case where the prediction model 25 predicts a plurality of candidates as information to be provided, the information providing device 100 determines whether there is other information that has not been proposed to the user yet. Thus, when the user does not agree, steps S24 to S26 are repeated until all the candidates are proposed to the user. Then, in a case where the user does not agree and all the candidates have been proposed to the user (step S24: No), the information providing processing ends.

Since medical information databases involve joining a plurality of tables, conditional search, and fuzzy search due to free-text input, if a query is issued after a user starts the operation and the search request is confirmed, it causes a delay in query generation and optimization, unnecessary expansion of the search range, and an increase in database load due to frequent occurrence of overlapping queries in routine tasks by the same user, resulting in issues such as screen response delay and waste of server resources.

In the present example embodiment, by narrowing down the database reference targets in advance based on the past logs and the access conditions of a user, and obtaining necessary information in advance or storing it in a session cache, it is possible to reduce the response delay and the database load when accessing medical information.

### Second Example Embodiment

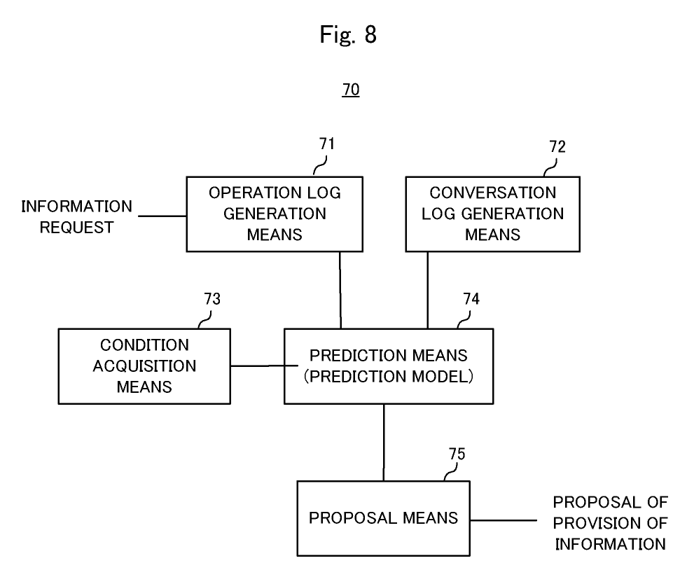

FIG. 8 is a block diagram illustrating a functional configuration of an information providing device according to a second example embodiment. An information providing device 70 includes an operation log generation means 71, a conversation log generation means 72, a condition acquisition means 73, a prediction means 74, and a proposal means 75.

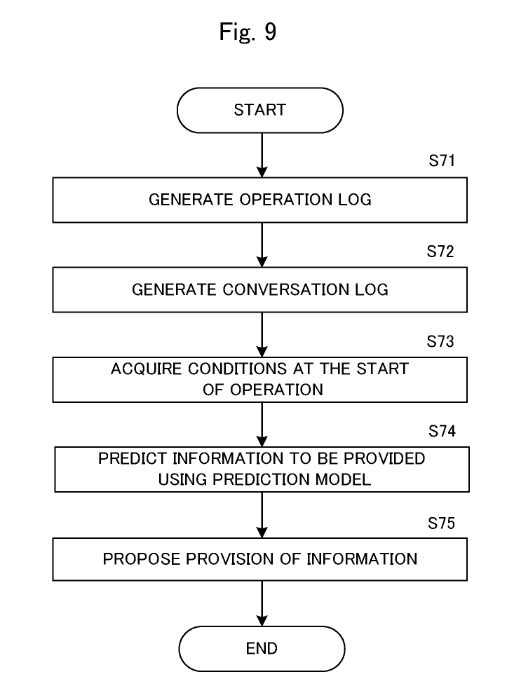

FIG. 9 is a flowchart of processing performed by the information providing device according to the second example embodiment. The operation log generation means 71 generates an operation log of the user in a case where the user accesses the medical information (step S71). The conversation log generation means 72 generates a conversation log between the user and an AI agent for supporting provision of the medical information (step S72). The condition acquisition means 73 acquires the conditions at the start of the operation by the user (step S73). The prediction means 74 predicts information to be provided to the user from the identification information of the user and the conditions at the start of the operation by the user using a prediction model trained in advance using the operation log and the conversation log (step S74). The proposal means 75 proposes, to the user, provision of the information to be provided (step S75).

According to the information providing device 70 of the second example embodiment, it is possible to predict and propose information required by a user according to the conditions in case where the user accesses a DB.

A part or all of the example embodiments described above may also be described as the following Supplementary notes, but not limited thereto.

### Supplementary note 1

An information providing device comprising:
an operation log generation means for generating an operation log of a user in a case where the user accesses medical information;
a conversation log generation means for generating a conversation log between the user and an AI agent that supports provision of medical information;
a condition acquisition means for acquiring a condition at a start of an operation by the user;
a prediction means for predicting information to be provided to the user from identification information of the user and the condition at the start of the operation by the user using a prediction model trained in advance using the operation log and the conversation log; and
a proposal means for proposing, to the user, provision of the information to be provided.

### Supplementary note 2

The information providing device according to Supplementary note 1, further comprising an information output means for outputting the information to be provided in a case where the user agrees with a proposal.

### Supplementary note 3

The information providing device according to Supplementary note 1, wherein the condition includes date and time of the operation by the user.

### Supplementary note 4

The information providing device according to Supplementary note 1, wherein the operation log includes the identification information of the user, date and time of the operation by the user, and information output to the user.

### Supplementary note 5

The information providing device according to Supplementary note 1, wherein the conversation log includes an information request in natural language input by the user, information requested by the user obtained by interpreting the information request in natural language, and information output to the user.

### Supplementary note 6

The information providing device according to Supplementary note 5, wherein the conversation log generation means interprets the information request in natural language using a language model to acquire the information requested by the user.

### Supplementary note 7

The information providing device according to Supplementary note 2, further comprising a database that stores the medical information,
wherein the information output means acquires the information to be provided from the database and outputs the information to be provided.

### Supplementary note 8

The information providing device according to Supplementary note 1,
wherein the prediction means predicts a plurality of pieces of information to be provided, and
wherein the proposal means outputs the pieces of information to be provided in descending order of prediction score.

### Supplementary note 9

An information providing method executed by a computer, the information providing method comprising:
generating an operation log of a user in a case where the user accesses medical information;
generating a conversation log between the user and an AI agent that supports provision of medical information;
acquiring a condition at the start of an operation by the user;
predicting information to be provided to the user from identification information of the user and the condition at the start of the operation by the user using a prediction model trained in advance using the operation log and the conversation log; and
proposing, to the user, provision of the information to be provided.

### Supplementary note 10

A program that causes a computer to execute processing comprising:
generating an operation log of a user in a case where the user accesses medical information;
generating a conversation log between the user and an AI agent that supports provision of medical information;
acquiring a condition at the start of an operation by the user;
predicting information to be provided to the user from identification information of the user and the condition at the start of the operation by the user using a prediction model trained in advance using the operation log and the conversation log; and
proposing, to the user, provision of the information to be provided.

Some or all of the configurations described in Supplementary Notes 2 to 8 dependent on the above-described Supplementary Note 1 can also be dependent on Supplementary Notes 9 and 10 by a dependency relationship similar to that of Supplementary Notes 2 to 8. Some or all of the configurations described as the Supplementary Notes can be similarly dependent on not only the Supplementary Notes 1, 9, and 10, but also diverse pieces of hardware and software, various recording means for recording software, or systems without departing from the above-described example embodiments.

While the present disclosure has been described with reference to the example embodiments and examples, the present disclosure is not limited to the above example embodiments and examples. Various changes which can be understood by those skilled in the art within the scope of the present disclosure can be made in the configuration and details of the present disclosure.

### DESCRIPTION OF SYMBOLS

11 Processor
21 Operation detection unit
22 Request interpretation model
23 Reference destination table detection model
24 Information output unit
25 Prediction model
40 RDB
41 Definition information
42 Table group
43 Operation log
44 Conversation log
100 Information providing device

1. An information providing device comprising:
a memory configured to store instructions; and
one or more processors configured to execute the instructions to:
generate an operation log of a user in a case where the user accesses medical information;
generate a conversation log between the user and an AI agent that supports provision of medical information;
acquire a condition at a start of an operation by the user;
predict information to be provided to the user from identification information of the user and the condition at the start of the operation by the user using a prediction model trained in advance using the operation log and the conversation log; and
propose, to the user, provision of the information to be provided.

2. The information providing device according to claim 1, wherein the one or more processors are further configured to execute the instructions to output the information to be provided in a case where the user agrees with a proposal.

3. The information providing device according to claim 1, wherein the condition includes date and time of the operation by the user.

4. The information providing device according to claim 1, wherein the operation log includes the identification information of the user, date and time of the operation by the user, and information output to the user.

5. The information providing device according to claim 1, wherein the conversation log includes an information request in natural language input by the user, information requested by the user obtained by interpreting the information request in natural language, and information output to the user.

6. The information providing device according to claim 5, wherein the one or more processors interpret the information request in natural language using a language model to acquire the information requested by the user.

7. The information providing device according to claim 2, further comprising a database that stores the medical information,
wherein the one or more processors acquire the information to be provided from the database and outputs the information to be provided.

8. The information providing device according to claim 1,
wherein the one or more processors predict a plurality of pieces of information to be provided, and
wherein the one or more processors output the pieces of information to be provided in descending order of prediction score.

9. An information providing method executed by a computer, comprising:
generating an operation log of a user in a case where the user accesses medical information;
generating a conversation log between the user and an AI agent that supports provision of medical information;
acquiring a condition at the start of an operation by the user;
predicting information to be provided to the user from identification information of the user and the condition at the start of the operation by the user using a prediction model trained in advance using the operation log and the conversation log; and
proposing, to the user, provision of the information to be provided.

10. A non-transitory computer-readable recording medium storing a program, the program causing a computer to execute processing comprising:
generating an operation log of a user in a case where the user accesses medical information;
generating a conversation log between the user and an AI agent that supports provision of medical information;
acquiring a condition at the start of an operation by the user;
predicting information to be provided to the user from identification information of the user and the condition at the start of the operation by the user using a prediction model trained in advance using the operation log and the conversation log; and
proposing, to the user, provision of the information to be provided.
